(19) United States
(12) Patent Application Publication
(10) Pub. No.: US 2007/0239023 A1
(43) Pub. Date: Oct. 11, 2007

(54) TRANSESOPHAGEAL ULTRASOUND PROBE WITH THIN AND FLEXIBLE WIRING

(76) Inventors: Harold M. Hastings, Garden City, NY (US); Edward Paul Harhen, Duxbury, MA (US)

Correspondence Address: PROSKAUER ROSE LLP;PATENT DEPARTMENT, 1585 BROADWAY, NEW YORK, NY, 10036-8299, US

(21) Appl. No.: 11/689,597

(22) Filed: Mar. 22, 2007

Related U.S. Application Data
(60) Provisional application No. 60/743,702, filed on Mar. 23, 2006.

(51) Int. Cl. A61B 8/14

(52) U.S. Cl. 600/459

(57) ABSTRACT

An ultrasound probe includes a flexible shaft with a channel that runs through the shaft in a proximal-distal direction, and an ultrasound transducer disposed in a housing mounted at the distal end of the shaft. Flexible wiring is disposed within the channel and is configured to carry signals to and from the transducer. The flexible wiring includes a plurality of substantially parallel conductors that are positioned above a ground plane, and separated from the ground plane by an insulating material. The substantially parallel conductors are also insulated from one another. In some embodiments, a grounded conductive shield is provided on the opposite side of the ground plane. Preferred approaches for implementing the flexible wiring include ribbon cable with a built-in ground plane and flexible printed circuit boards (i.e., flex circuits).

### CROSS REFERENCE TO RELATED APPLICATIONS

This application claims the benefit of U.S. provisional application 60/743,702, filed Mar. 23, 2006.

### BACKGROUND

Conventional TEE (transesophageal echocardiography) probes employ an ultrasound transducer with a large number of active elements (e.g., 64 elements) at the distal end of the probe. Excitation signals to the transducer and return signals from the transducer are typically carried between a connector at the proximal end of the probe and the transducer at the distal end of the probe via a bundle of mini-coax cables that run through the center of the probe, with one mini-coax cable dedicated to each element of the transducer. This arrangement provides excellent shielding for the various signals traveling up and down through the probe.

These conventional probes typically measure between 10 and 15 mm in diameter at the transducer end. Because each element uses own mini-coax cable, the channel that runs through the center of the probe must be large enough to house the correspondingly large number of mini-coax cables. For example, to accommodate a 64 element transducer, the probe would need a bundle of 64 mini-coax cables running down its center. With this construction, the probe's body can become relatively thick and its flexibility may decrease. The resulting probes are also relatively expensive.

The thickness and stiffness of the probe's shaft is not usually a limiting factor when the transducer itself has a large diameter. However, when using a transducer with a small diameter (e.g., on the order of 5 mm, as described in U.S. patent application Ser. No. 10/996,816, filed Nov. 24, 2004, and entitled “Transesophageal Ultrasound Using a Narrow Probe,” which is incorporated herein by reference), the thickness and stiffness of the probe's shaft can become a limiting factor in certain circumstances.

### SUMMARY OF THE INVENTION

Flexible wiring is used to carry signals to and from the transducer at the distal end of an ultrasound probe. The flexible wiring includes a plurality of substantially parallel conductors that are positioned above a ground plane, and separated from the ground plane by an insulating material. The substantially parallel conductors are also insulated from one another. In some embodiments, the flexible wiring is implemented using ribbon cable with a built-in ground plane. In other embodiment, the flexible wiring is implemented using flexible printed circuit boards (i.e., flex circuits).

### BRIEF DESCRIPTION OF THE DRAWINGS

FIG. 1 is a schematic representation of an ultrasound probe connected to an ultrasound imaging machine.

FIG. 2 depicts a first preferred embodiment of the invention that is made from a ribbon cable with a built-in ground plane, plus a conductive shield.

FIG. 3 depicts a second preferred embodiment of the invention that is made from a ribbon cable with a built-in ground plane, plus a conductive shield.

FIG. 4 depicts a third preferred embodiment of the invention that is made from a ribbon cable with a two built-in ground planes.

FIG. 5 depicts a fourth preferred embodiment of the invention that is similar to the third embodiment, in which the first and last wires are grounded.

FIG. 6 depicts a fifth preferred embodiment of the invention that is made from a stack of ribbon cables with a built-in ground planes.

FIG. 7 depicts a sixth preferred embodiment of the invention that is made using a flexible printed circuit board with a two built-in ground planes.

FIG. 8 depicts a seventh preferred embodiment of the invention that is similar to the sixth embodiment, in which the first and last conductors are grounded.

### DETAILED DESCRIPTION OF THE PREFERRED EMBODIMENTS

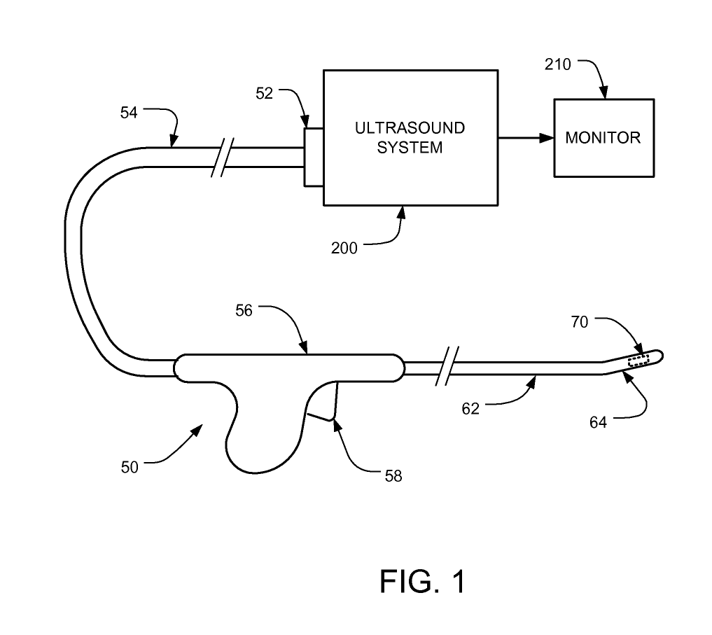

FIG. 1 depicts an ultrasound probe 50 that is connected to an ultrasound system 200. An ultrasound transducer 70 is located in a transducer housing 64 at the distalmost portion of the probe 50. The ultrasound transducer 70 preferably has at least 24 active elements and preferably has 44 or fewer active elements. More preferably, it has either 32 or 36 active elements. The next portion (moving in the proximal direction) is the flexible shaft 62, which is positioned between the transducer housing 64 and the handle 56. This shaft 62 should be flexible enough so that the transducer housing 64 can be positioned past the relevant anatomical structures to the desired location, and the handle 56 facilitates the positioning of the transducer housing 64 by the operator. The flexible shaft 62 may be made with a uniform flexibility throughout its entire length. In alternative embodiments the flexible shaft 62 may be made with one or more subsections that are more flexible than the remainder of the flexible shaft 62, and/or with one or more less flexible or non-flexible subsections. Optionally, the handle 56 may contain a triggering mechanism 58 which the operator uses to bend a distal portion of the probe 50 to a desired anatomical position.

At the other end of the handle 56 is a cable 54, which is connected to the ultrasound system 200 so that the ultrasound system 200 can operate the probe. Signals from the ultrasound system 200 travel through the probe 50 via appropriate wiring and any intermediate circuitry (not shown) to drive the transducer 70, and return signals from the transducer 70 similarly travel back through the probe 50 to the ultrasound system 200 where they are ultimately processed into images. The images are then displayed on the monitor 210 in a manner well known to persons skilled in the relevant art. To use the probe, the distal portion of the probe 50 is passed through the appropriate orifice (e.g., the mouth or nose) until the transducer 70 is positioned at the desired anatomical location (e.g., the fundus of the stomach), with the proximal end of the shaft 62 protruding out of the patient's body via the orifice. Images are then captured.

The inventors have come up with a number of approaches for reducing the cross section of the probe's wiring and for improving the flexibility of the wiring to obtain corresponding improvements in the shaft 62. All other portions of the probe besides the wiring that is used in the shaft 62 (including but not limited to the handle, the articulation controls, the interface cable, the connector, the bending mechanism, etc.) may be constructed using any of a variety of conventional techniques. However, the size of the probe 50 is preferably scaled down to take advantage of the reduced cross section of the wiring.

A first set of embodiments described below use a ribbon cable to carry the relevant signals to and from the ultrasound transducer instead of the bundle of mini-coax cables that was used in the prior art TEE cables. Note that as used herein, the phrase “to carry signals to and from the transducer” includes the situation where the conductors are connected directly to the transducer elements, and also includes the situation where one or more components (e.g., passive components, receive amplifiers, etc.) are interposed between the conductors and the transducer, wherein the interposed components are located in the vicinity of the transducer 70.

One suitable type of ribbon cable for this application is micro-miniature ribbon cable made by Gore™ (i.e., W. L. Gore & Associates in Newark, Del.), which has a set of parallel wires and a ground plane positioned beneath the wires. However, even when ribbon cable with a ground plane (hereinafter “RCGP”) is used, the shielding may not be adequate, in which case additional shielding should be provided. One option (not shown) is to surround the RCGP with a grounded jacket made of braided metal or mesh metal, or to surround the RCGP with grounded metal foil. Optionally, the jacket, mesh, or foil may be surrounded by a suitable protective sheath. The RCGP and the surrounding grounding material is then threaded through the center channel of the probe 50 and wired up to carry the signals between the connector 52 at the proximal end of the probe and the transducer 70 at the distal end of the probe. One example of a probe body with a center channel that is suitably shaped for holding the RCGP is depicted in U.S. application Ser. No. 11/681,837, filed Mar. 6, 2007, which is incorporated herein by reference.

In alternative embodiments, instead of running the RCGP all the way from the transducer 70 to the connector 52, the RCGP is only used in the transducer housing 64 and the shaft 62. The proximal end of the RCGP is then connected to conventional wiring, which carries the signals along the remainder of the path to the connector 52. A convenient place to make the connection between the RCGP and the conventional wiring is in the handle 56. This connection may be implemented by hard-wiring the two types of wiring together, or alternatively using a suitable connectorized interface. Examples of this type of connectorized interface are disclosed in U.S. application Ser. No. 11/279,510, filed Apr. 12, 2006, which is incorporated herein by reference.

The embodiments described below in connection with FIGS. 1-5 also use RCGP, but achieve further reduction in thickness as compared to using RCGP that is completely surrounded by grounded material.

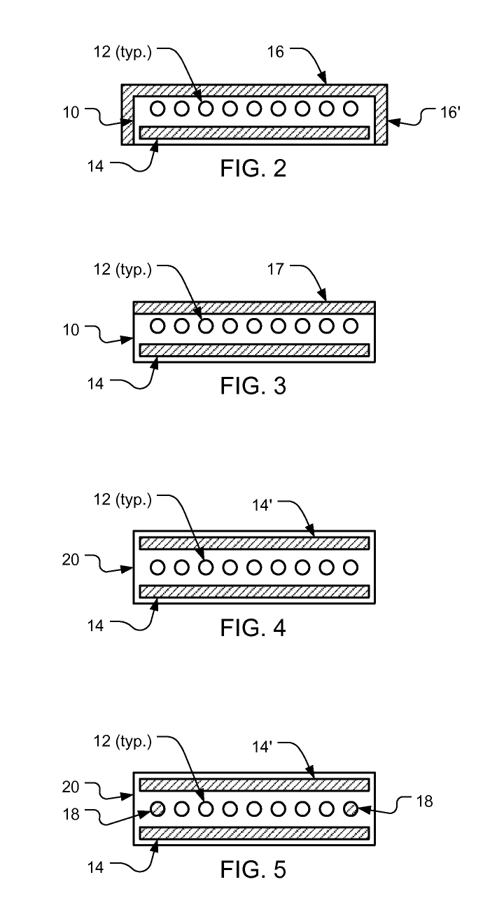

FIG. 2 depicts one preferred embodiment of the invention, shown in cross section. The RCGP 10 contains a plurality of wires 12. A ground plane 14 is provided on one side of the RCGP 10, and a thin grounded conductive shield 16 (e.g., copper foil) is added on the opposite side of the RCGP. The thin conductive shield 16 in this embodiment also has sidewalls 16′ that extend down over the side edges of the RCGP 10. The ground plane 14 and the thin conductive shield 16 are preferably wired together (i.e., electrically connected) and grounded e.g., adjacent to the transducer and/or at the proximal end of the probe.

FIG. 3 depicts another preferred embodiment of the invention, shown in cross section. This embodiment is similar to the FIG. 2 embodiment, except that the thin conductive shield 17 does not have the sidewalls that extend over the side edges of the RCGP 10. The ground plane 14 and the thin conductive shield 16 are preferably wired together and grounded e.g., adjacent to the transducer and/or at the proximal end of the cable.

The thin conductive shield 16, 17 shown in the FIG. 2 and FIG. 3 embodiments may be added onto an off-the-shelf RCGP (e.g., by affixing a piece of metal foil to a conventional RCGP 10 using a suitable adhesive, or by painting the outside of a conventional RCGP with conductive paint). Alternatively, the thin conductive shield may be integrated into the RCGP itself by integrating a second ground plane 14′ into a custom-designed RCGP 20, as shown in FIG. 4.

FIG. 5 depicts another preferred embodiment of the invention, shown in cross section. This embodiment is similar to the FIG. 4 embodiment, except that the last wire 18 on either edge of the RCGP 20 is grounded, and is not used to transmit signals. The ground planes 14, 14′ and the wires 18 on either edge of the RCGP are preferably wired together and grounded e.g., adjacent to the transducer and/or at the proximal end of the cable. Grounding the edge wires 18 provides additional shielding at the edge of the RCGP. Note that the grounded edge wires 18 shown in this embodiment may also be added to the FIG. 3 embodiment to provide a similar shielding effect.

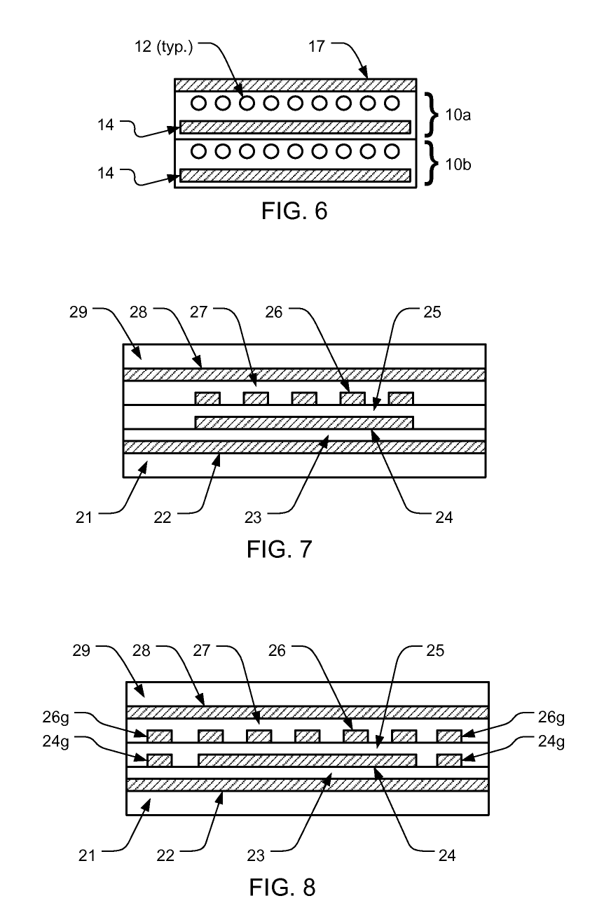

Optionally, any of the embodiments described herein may be modified by adding one or more additional layers of conventional RCGP beneath the RCGP 10, 20 depicted in FIGS. 1-4, so that each set of signal wires 12 has a ground plane 14 below and either a ground plane 14′ or a thin conductive shield 16, 17 above. For example, FIG. 6 shows the RCGP 10a covered by a thin conductive shield 17 as in the FIG. 3 embodiment, but with a second layer of conventional RCGP 10b positioned beneath the first RCGP 10a. With this configuration, each wire 12 is either sandwiched between two ground planes 14 or sandwiched between one ground plane 14 and the thin conductive shield 17. All the ground planes are preferably wired together and grounded e.g., adjacent to the transducer and/or at the proximal end of the cable. Note that the grounded end wires described above in connection with the FIG. 5 embodiment may optionally be added to this embodiment as well.

In the above-described embodiments, a variety of different configurations may be used to connect the RCGP to the ultrasound transducer. In one configuration, two wires are used for each element of the ultrasound transducer—one for the signal, and one for the signal return. Optionally, selected wires within the ribbon cable may be grounded to prevent crosstalk (e.g., by grounding every other wire). In a second configuration, only one wire is used for each element of the transducer, and all the elements all share a common return. With this configuration, the shield 14 (or 14′) is preferably used as the common return. Note that while only nine wires 12 are shown in FIGS. 2-5 for clarity, in practice a larger number of wires will be needed to interface with the ultrasound transducer. For example, with the second configuration, 32 wires would be needed to interface with a 32 element transducer.

A second set of embodiments are similar to the first set of embodiments described above, except that a flexible printed circuit board is used to carry the relevant signals to and from the transducer (instead of the ribbon cables described above in connection with FIGS. 2-6). The manufacturing process for flexible printed circuit boards (also known as flex circuits) is well known to persons skilled in the relevant arts.

FIG. 7 depicts a cross section of a multi-layer flexible printed circuit board that is suitable for carrying signals to and from the transducer. The bottom layer 21 is an insulating substrate. The next layer is a conductive ground plane 22, followed by an insulating layer 23. The next layer is a wide conductor 24, followed by another insulating layer 25. The next layer has a plurality of parallel signal traces 26, followed by another insulating layer 27. The next layer is another conductive ground plane 28, followed by the top insulating layer 29. In the illustrated embodiment, each signal travels over its own trace 26, and all the return signals use the wide conductor 24 as a common signal return. In alternative embodiments (not shown), the wide conductor 24 may be replaced with a set of individual traces, one for each signal.

Note that although only five signal traces are shown in FIG. 7 for clarity, there will actually be one signal trace for each element of the ultrasound transducer. For example, 32 traces would be needed to interface with a 32 element transducer. The ground planes 22, 28 may be grounded at the proximal and/or the distal end. All the layers 21-29 are flexible. Polyimide (e.g., Kapton) is a suitable material for the insulating layers and the substrates. The intermediate insulating layers are preferably at least 0.001 inches thick. Since increasing the thickness reduces the capacitance, increasing the thickness of the insulating layers is desirable. While current popular manufacturing processes may limit the thickness (e.g., to 0.004 inches), the use of thicker layers is contemplated as other manufacturing processes become available.

FIG. 8 is similar to FIG. 7, except that grounded traces 26g are added at both lateral ends of the set of signal traces 26, and grounded traces 24g are added at both lateral ends of the wide conductor 24 to provide additional shielding. Each of these traces 24g, 26g may be grounded at either the proximal and/or the distal end.

As in the RCGP embodiments described above in connection with FIGS. 2-6, a variety of different configurations may be used to connect the traces 26 to the ultrasound transducer. In one configuration, two traces are used for each element of the ultrasound transducer—one for the signal, and one for the signal return. In a second configuration, only one trace is used for each element of the transducer, and all the elements all share a common return. In one preferred embodiment of this configuration, the wide conductor 24 is used as the common return. In another preferred embodiment of this configuration, the wide conductor 24, the end conductors 24g, and the insulating layer 25 are all omitted, and one of the ground planes 22, 28 is used as the common return.

In certain circumstances, additional grounding may be required to adequately reduce RF signal levels on nominally grounded components in any of the above-described embodiments. As is well-known, good grounding may be achieved by using a very short ground lead to a good earth ground. In many cases, however, this may not be practical, e.g., when a good earth ground is not available or when longer signal paths are required for device functioning. For example, when a 2.4 meter long cable is used (assuming a nominal 6 MHz signal and a typical velocity factor of 0.67), the inductance of the shield can have significant effects on noise. However, when the wires are less than one quarter effective wavelength long, a capacitor (e.g., a ceramic disc capacitor, not shown) may be wired in series with the shield to cancel out series inductance and make the lines behave electrically as if they were very short. This may be done in one or more locations along the signal path, as required (e.g., at the transducer end of the wiring or at the handle end of the wiring). Optionally, an inductor (e.g., an RF choke, not shown) may be wired in parallel with this capacitor to preserve the DC ground.

The embodiments described above produce wiring that is thinner and more flexible than the prior art, while still providing adequate shielding for TEE applications. This helps make the TEE probes thinner and more flexible. Note that while the invention is described above in the context of transesophageal ultrasound probes, it may also be used in other contexts that can benefit from the reduced size and improved flexibility (e.g., ultrasound probes configured for insertion into different locations in the body or ultrasound probes configured for nonmedical applications). The invention may even be applied outside the context of ultrasound probes in situations where size must be minimized, but flexibility and shielding must be maintained.

We claim:

1. An ultrasound probe comprising:
a flexible shaft having a proximal end and a distal end, with a channel that runs through the shaft in a proximal-distal direction;
a housing for an ultrasound transducer positioned at the distal end of the shaft;
an ultrasound transducer mounted in the housing; and
flexible wiring disposed within the channel and configured to carry signals to and from the transducer, wherein the flexible wiring comprises a plurality of substantially parallel conductors that are insulated from one another and disposed above a first ground plane, with each of the substantially parallel conductors separated from the first ground plane by an insulating material.

2. The ultrasound probe of claim 1, wherein the flexible wiring further comprises a grounded conductive shield disposed above the plurality of substantially parallel conductors, with each of the substantially parallel conductors separated from the conductive shield by an insulating material.

3. The ultrasound probe of claim 2, wherein the flexible wiring comprises at least 24 substantially parallel conductors that are insulated from one another and disposed above the first ground plane.

4. The ultrasound probe of claim 1, wherein the flexible wiring further comprises a grounded conductive shield disposed above the plurality of substantially parallel conductors and also disposed on each lateral side of the plurality of substantially parallel conductors, with each of the substantially parallel conductors separated from the conductive shield by an insulating material.

5. The ultrasound probe of claim 4, wherein the flexible wiring comprises at least 24 substantially parallel conductors that are insulated from one another and disposed above the first ground plane.

6. The ultrasound probe of claim 1, wherein the flexible wiring further comprises a grounded conductive shield disposed above the plurality of substantially parallel conductors, with each of the substantially parallel conductors separated from the conductive shield by an insulating material, and wherein the conductors located at each lateral end of the plurality of substantially parallel conductors are grounded.

7. The ultrasound probe of claim 6, wherein the flexible wiring comprises at least 24 substantially parallel conductors that are insulated from one another and disposed above the first ground plane.

8. An ultrasound probe comprising:
a flexible shaft having a proximal end and a distal end, with a channel that runs through the shaft in a proximal-distal direction;
a housing for an ultrasound transducer positioned at the distal end of the shaft;
an ultrasound transducer mounted in the housing; and
a first flexible ribbon cable disposed within the channel and configured to carry signals to and from the transducer, wherein the first flexible ribbon cable comprises a plurality of substantially parallel conductors that are insulated from one another and disposed above a first ground plane, with each of the substantially parallel conductors separated from the first ground plane by an insulating material.

9. The ultrasound probe of claim 8, wherein the first flexible ribbon cable further comprises a second ground plane disposed above the plurality of substantially parallel conductors, with each of the substantially parallel conductors separated from the second ground plane by an insulating material.

10. The ultrasound probe of claim 9, wherein the first flexible ribbon cable comprises at least 24 substantially parallel conductors that are insulated from one another and disposed above the first ground plane.

11. The ultrasound probe of claim 9, wherein the first and last conductors of the first flexible ribbon cable are grounded.

12. The ultrasound probe of claim 8, further comprising a second flexible ribbon cable disposed within the channel and configured to carry signals to and from the transducer, wherein the second flexible ribbon cable comprises a plurality of substantially parallel conductors that are insulated from one another and disposed above a second ground plane, with each of the substantially parallel conductors separated from the second ground plane by an insulating material, and wherein second flexible ribbon cable is oriented with respect to the first flexible ribbon cable so that the second ground plane is disposed between the substantially parallel conductors of the first flexible ribbon cable and the substantially parallel conductors of the second flexible ribbon cable.

13. An ultrasound probe comprising:
a flexible shaft having a proximal end and a distal end, with a channel that runs through the shaft in a proximal-distal direction;
a housing for an ultrasound transducer positioned at the distal end of the shaft;
an ultrasound transducer mounted in the housing; and
a flexible printed circuit board disposed within the channel and configured to carry signals to and from the transducer, wherein the flexible printed circuit board comprises a plurality of substantially parallel conductors that are insulated from one another and disposed above a first ground plane, with an insulating material disposed between the plurality of substantially parallel conductors and the first ground plane.

14. The ultrasound probe of claim 13, wherein the flexible printed circuit board further comprises a second ground plane disposed above the plurality of substantially parallel conductors, with an insulating material disposed between the plurality of substantially parallel conductors and the second ground plane.

15. The ultrasound probe of claim 14, wherein the first insulating material and the second insulating material are each at least 0.001 inches thick.

16. The ultrasound probe of claim 14, wherein the first insulating material and the second insulating material comprise polyimide.

17. The ultrasound probe of claim 14, wherein the first insulating material and the second insulating material comprise Kapton.

18. The ultrasound probe of claim 14, wherein the flexible printed circuit board comprises at least 24 substantially parallel conductors that are insulated from one another and disposed above the first ground plane.

19. The ultrasound probe of claim 18, wherein the first insulating material and the second insulating material comprise polyimide and are each at least 0.001 inches thick.

20. The ultrasound probe of claim 14, wherein the first and last conductors of the plurality of substantially parallel conductors are grounded.

21. The ultrasound probe of claim 14, wherein a common return conductor is disposed between, and insulated from, the plurality of substantially parallel conductors and the first ground plane.

22. The ultrasound probe of claim 21, wherein the first and last conductors of the plurality of substantially parallel conductors are grounded.
